(19) United States
(12) Patent Application Publication
(10) Pub. No.: US 2003/0009193 A1
(43) Pub. Date: Jan. 9, 2003

(54) Surgical instrument

(76) Inventor: Santi Corsaro, Cincinnati, OH (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/132,500

(22) Filed: Apr. 25, 2002

(30) Foreign Application Priority Data
May 2, 2001 (DE) GFR 101 21 305.0

(51) Int. Cl. A61B 17/28

(52) U.S. Cl. 606/207; 227/175.1

(57) ABSTRACT

A surgical instrument (200) comprises a frame (210) and a jaw assembly (220) in the distal region of the instrument (200). The jaw assembly (220) has a generally straight first tissue interface (230), a generally straight second tissue interface (232) and a tissue effector (e.g., one or more generally straight rows of staples and/or a knife). The jaw assembly (220) is adapted to grip tissue of a patient between the first tissue interface (230) and the second tissue interface (232) and to penetrate the gripped tissue by the tissue effector. The first tissue interface (230) can be moved relative to the second tissue interface (232) by means of a moving device. An actuating device is adapted to actuate the tissue effector. Both the first tissue interface (230) and the second tissue interface (232) have a generally wavy shape, the crests and valleys of the first tissue interface (230) generally matching the valleys and crests, respectively, of the second tissue interface (232).

### FILED OF THE INVENTION

The invention relates to a surgical instrument having a jaw assembly with a generally straight first tissue interface, a generally straight second tissue interface and a tissue effector. This instrument can be designed as a stapling instrument and/or a cutting instrument.

### BACKGROUND OF THE INVENTION

Instruments of this kind are widely used in surgery. Generally, the surgical instrument comprises a frame having a body portion and a handle and, in the distal region of the instrument, a jaw assembly. The jaw assembly has a generally straight design with a generally straight first tissue interface and a generally straight second tissue interface, the jaw assembly being adapted to grip tissue of a patient between the first tissue interface and the second tissue interface. Moreover, a tissue effector for penetrating the gripped tissue is provided. An example of a tissue effector is a set of staples which are arranged in the jaw assembly and, upon actuation of the instrument, exit from the first tissue interface and penetrate the tissue, until their ends are formed or bent by the second tissue interface. Another example is a knife for cutting the tissue when the instrument is actuated. The knife can be used in combination with the staples, but it can also be arranged in a pure cutting instrument.

U.S. Pat. No. 4,527,724 discloses a linear surgical stapling instrument in which a straight jaw assembly is located at the distal end of a shaft. The longitudinal axis of the jaw assembly extends transversely with respect to the longitudinal axis of the shaft.

U.S. Pat. No. 4,633,874 describes a surgical stapling instrument in which the longitudinal axis of the jaw assembly coincides with the longitudinal axis of the rest of the instrument. A pusher bar and knife blade assembly is slidable longitudinally relative to the jaw members to sequentially drive staples from a cartridge and form the staples against one of the tissue interfaces (which is designed as an anvil) to produce a pair of laterally spaces rows of formed staples in tissue gripped between the jaw members (i.e. between the first tissue interface and the second tissue interface) and to cut the tissue along a line between the staple rows.

Another straight or linear stapling instrument in which the longitudinal axis of the jaw assembly runs along the longitudinal axis of the whole instrument is disclosed in U.S. Pat. No. 5,307,976. This document also describes details of the actuating components and safety means of the instrument.

In all these surgical instruments, the tissue interfaces, i.e. those parts of the jaw assembly which contact the patient's tissue to be effected (e.g., stapled and/or cut), are designed as generally flat surfaces. This holds for instruments used in open surgery and in endoscopic surgery. In straight (linear) cutters for open or endoscopic surgery, the tissue interfaces usually extend along the longitudinal axis of the instrument. For linear staplers applied in open surgery, the tissue interfaces usually are arranged transversally with respect to the longitudinal axis of the instrument. In the current designs of these kinds of surgical instruments, the jaw assembly is generally longer than the length of tissue which can be gripped between the first tissue interface and the second tissue interface. This can make it difficult to manipulate and utilize the instrument in certain situations, in particular in space-limited anatomical regions.

It is the object of the invention to provide a surgical instrument of the above-described kind which is able to effect tissue in a more space-saving manner.

This problem is solved by a surgical instrument having the features of claim 1. Advantageous embodiments of the invention follow from the dependent claims. Claims 19 to 21 are directed to components of the surgical instrument.

### SUMMARY OF THE INVENTION

The surgical instrument according to the invention comprises a frame having a body portion and a handle, as well as a jaw assembly in the distal region of the instrument. The jaw assembly has a generally straight first tissue interface, a generally straight second tissue interface and a tissue effector, and the jaw assembly is adapted to grip tissue of a patient between the first tissue interface and the second tissue interface and to penetrate the gripped tissue by the tissue effector. A moving device is adapted to move the first tissue interface relative to the second tissue interface. An actuating device is adapted to actuate the tissue effector. According to the invention, the first tissue interface and the second tissue interface have a generally wavy shape, the crests and valleys of the first tissue interface generally matching the valleys and crests, respectively, of the second tissue interface.

In other words, the tissue interfaces are not flat, as in conventional instruments, but have a three-dimensional, wavy shape along the length of the jaw assembly, e.g. a sinusoidal shape. Thus, the path length following the valleys and crests of the wavy shape is greater than the linear extension of the tissue interfaces. Generally, this allows the instrument to effect a length of tissue greater than the length of the jaw assembly. If the surgical instrument is a stapling instrument and/or a cutting instrument, a staple line and/or cutting line can be created which follows the shape of the tissue gripped between the first tissue interface and the second tissue interface, i.e. the generally wavy shape of the tissue interfaces, and which is effectively longer than the staple line or the cutting line of a conventional instrument. The greater length of the staple line or cutting line has advantages, e.g., for a given length of a staple line or cutting line, the jaw assembly of the surgical instrument according to the invention can be smaller than the jaw assembly of a conventional instrument. Thus, the invention allows for the design of smaller or shorter and less bulky jaw assembly components that will facilitate easier access to anatomic sites.

As already mentioned, the surgical instrument can be a stapling instrument. In this case, the jaw assembly includes a cartridge device having the first tissue interface and an anvil having the second tissue interface. The tissue effector comprises, associated to the cartridge device, at least one generally straight row of staples which, upon actuation of the actuating device, exit from the first tissue interface, the second tissue interface being adapted to form the ends of the staples. Preferably, the line defining the row of staples follows the wavy shape of the first tissue interface. Alternatively, e.g., the staples could be arranged at the top of the crests and/or at the bottom of the valleys of the first tissue interface.

In an advantageous version of a stapling instrument, the tissue effector comprises a knife, which is contained in the cartridge device, runs generally in parallel to the row of staples and, upon actuation of the actuating device, moves towards the anvil. The knife can have a wavy cutting edge, which, preferably, generally runs in parallel to the wavy shape of the first tissue interface.

In another advantageous version of a stapling instrument, the tissue effector comprises a knife, which is adapted to move, upon actuation of the actuating device, from one end region of the cartridge device to the opposite end region of the cartridge device, generally in parallel to and along the row of staples, in order to sequentially cut the tissue gripped between the first tissue interface and the second tissue interface.

Preferably, in a stapling instrument comprising a knife, there is at least one row of staples on each side of the knife. This allows opposing layers of tissue to be connected at both sides of the knife, i.e. to create two closed tissue parts which are separated when the knife is actuated.

In advantageous versions of a stapling instrument, the anvil is removable, and the cartridge device comprises a removable cartridge containing the staples.

The surgical instrument according to the invention can also be designed as a cutting instrument (without stapling function). In this case, the jaw assembly includes a first gripping jaw having the first tissue interface and a second gripping jaw having the second tissue interface. The tissue effector comprises a knife, which is adapted to cut, upon actuation of the actuating device, the tissue gripped between the first tissue interface and the second tissue interface.

In a preferred version of a cutting instrument, the knife is generally straight, generally extends along the jaw assembly, and, upon actuation of the actuating device, moves from the first tissue interface towards the second tissue interface. In this case, the knife can have a wavy cutting edge, which, preferably, generally runs in parallel to the wavy shape of the first tissue interface.

In another design of a cutting instrument, the knife is adapted to move, upon actuation of the actuating device, from one end region of the gripping jaws to the opposite end region of the gripping jaws, generally along the gripping jaws, in order to sequentially cut the tissue gripped between the first tissue interface and the second tissue interface.

In advantageous versions of a stapling instrument and/or a cutting instrument, the moving device is adapted to move the first tissue interface relative to the second tissue interface in a generally parallel relationship. This design facilitates insertion of the tissue into the space between the first tissue interface and the second tissue interface when these interfaces are in the spaced apart state.

Preferably, the body portion of the frame of the surgical instrument includes a shaft. This allows for two basic geometries of the instrument. In one of these geometries, the longitudinal axes of the first tissue interface and the second tissue interface run transversally with respect to the longitudinal axis of the shaft. This design is advantageous for applications in open surgery. When the instrument includes a knife, a generally straight knife which generally extends along the jaw assembly allows for a mechanically simple actuating device. In the other basic geometry, the longitudinal axes of the first tissue interface and the second tissue interface run generally in parallel with respect to the longitudinal axis of the shaft. This geometry is advantageous for endoscopic applications, and a sequentially cutting knife generally involves a simple design of the actuating device.

The jaw assembly can be removably mounted in the distal end region of the body portion. This allows for a design of the surgical instrument in which the jaw assembly is disposable, whereas the rest of the instrument, which is not contaminated or only slightly contaminated in the surgical procedure and which includes many components of the moving device and the actuating device, can be re-used after sterilization.

Apart from the three-dimensional shape of the tissue interfaces and any adaptions caused thereby, the surgical instrument according to the invention is generally designed as a conventional instrument. That means, it includes components like a handle, a shaft, gripping jaws or an anvil and a cartridge device, mechanical members of the moving device and the actuating device, safety elements like locking means against unintentional operation, and so on, which are generally known from the prior art, e.g., U.S. Pat. Nos. 4,527,724, 4,633,874, or 5,307,976. Moreover, the surgical instrument according to the invention can be used in a similar way as the prior art instruments.

### DETAILED DESCRIPTION OF THE DRAWINGS

In the following, the surgical instrument according to the invention is further explained by means of embodiments. The drawings show in

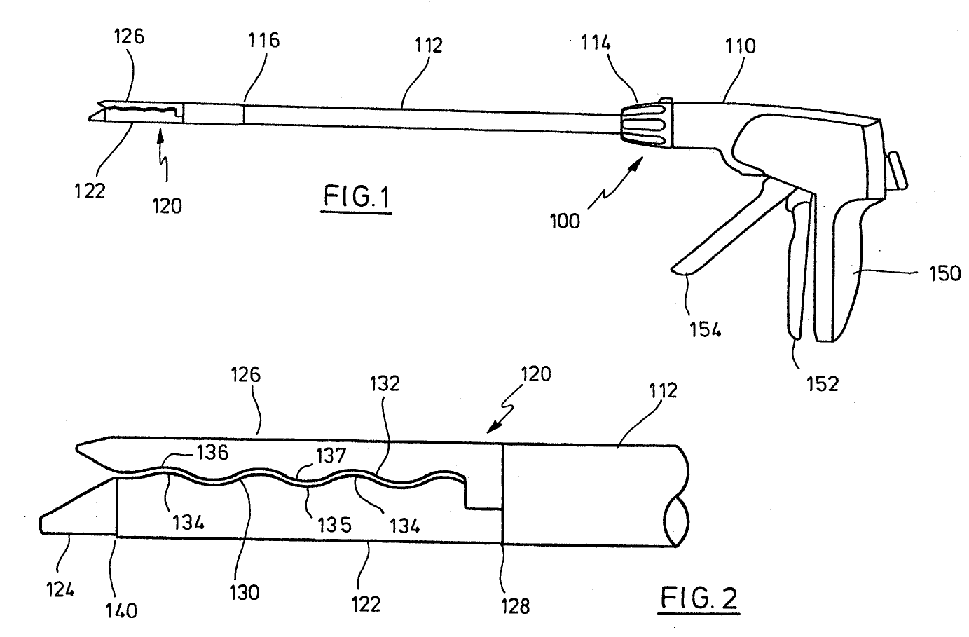

FIG. 1 a side view of a first embodiment of the surgical instrument according to the invention,

FIG. 2 a magnified side view of the jaw assembly of the instrument of FIG. 1,

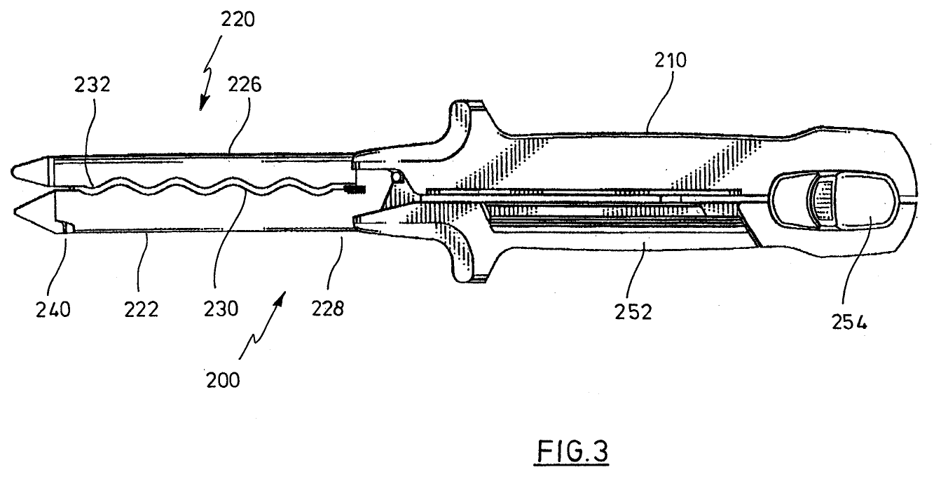

FIG. 3 a side view of a second embodiment of the surgical instrument according to the invention,

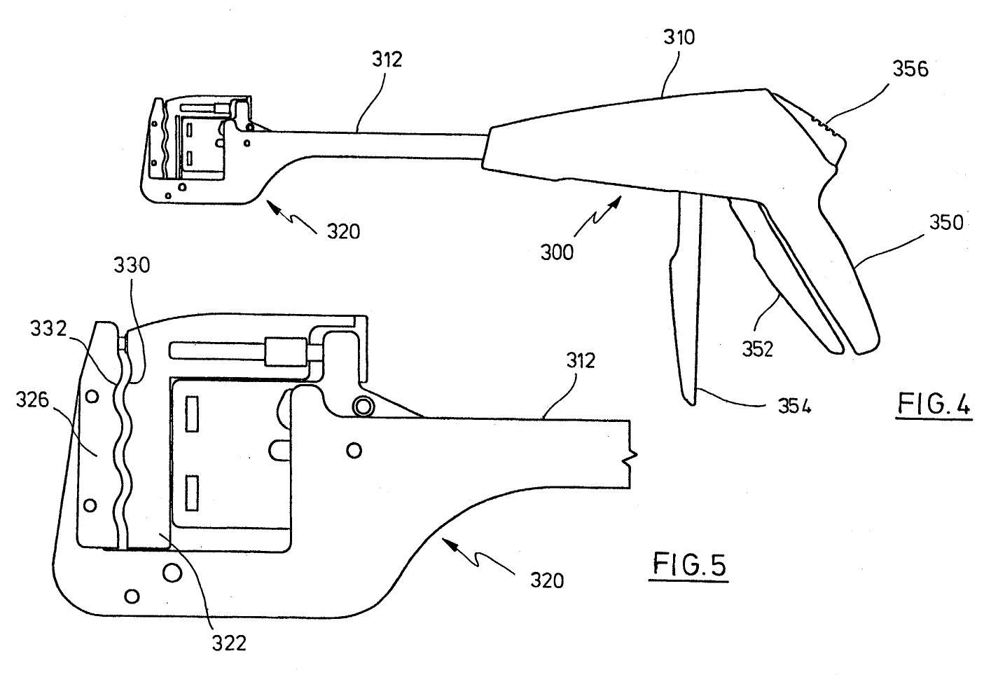

FIG. 4 a side view of a third embodiment of the surgical instrument according to the invention, and

FIG. 5 a magnified side view of the jaw assembly of the instrument of FIG. 4.

### DETAILED DESCRIPTION OF THE INVENTION

FIG. 1 illustrates a surgical instrument 100 which is designed as a linear stapler and can be used in endoscopic applications. This general type of instrument is known, e.g. from U.S. Pat. No. 5,307,976.

The instrument 100 comprises a handle section 110 and a shaft 112, which is rotatably connected to the handle section 110 via rotating means 114. The handle section 110 and the shaft 112 form a body portion.

At the distal end 116 of the shaft 112, a jaw assembly 120 is mounted in a removable manner. FIG. 2 is a magnified view of the jaw assembly 120. The jaw assembly 120 includes a cartridge device 122 (lower jaw in FIGS. 1 and 2) and an anvil 126 (upper jaw in FIGS. 1 and 2). The anvil 126 is swivellably mounted in the jaw assembly 120, but when the anvil 126 is close to the cartridge device 122, it moves in a parallel relationship with respect to the cartridge device 122.

The cartridge device 122 comprises a removable cartridge 124, which essentially extends along the length of the cartridge device 122. FIG. 2 shows the distal end region of the cartridge 124 which protrudes from a mounting base provided in the cartridge device 122. In the embodiment, the cartridge 124 contains six parallel straight rows of staples in which staples of adjacent rows are staggered with respect to each other. The ends of the staples point towards the anvil 126. The surface of the cartridge device 122 including the cartridge 124 opposite to the anvil 126 is called first tissue interface 130.

Similarly, the surface of the anvil 126 opposite to the cartridge device 122 is called second tissue interface 132. The second tissue interface comprises six parallel rows of depressions, which match to the positions of the staples in the cartridge 124 and are designed to form the pointed ends of the staples when they are expelled from the cartridge 124 upon actuation of the instrument 100.

The features of the instrument 100 described so far are those of a conventional stapler, and further details of the embodiment can be found in U.S. Pat. No. 5,307,976.

Whereas in a conventional stapler the first tissue interface and the second tissue interface are essentially flat or planar, the first tissue interface 130 of the instrument 100 has a generally wavy shape with crests 134 and valleys 135, see FIG. 2. The second tissue interface 132 has a generally wavy shape as well, and its valleys 136 and crests 137 match to the crests 134 and valleys 135, respectively, of the first tissue interface 130, see FIG. 2.

The proximal area of the instrument 100 is designed in a conventional manner, see U.S. Pat. No. 5,307,976 for more details on the embodiment. In summary, a handle 150 emerging from the handle section 110 serves for holding the instrument 100. A lever 152 is a component of a moving device for moving the first tissue interface 130 relative to the second tissue interface 132. That means, when the lever 152 is swivelled towards the handle 150, the anvil 126 moves towards the cartridge device 122 (i.e., the jaws of the jaw assembly 120 close), the force being transmitted by further components in the handle section 110, the shaft 112 and the jaw assembly 120, as explained, e.g., in U.S. Pat. No. 5,307,976.

A firing trigger 154 is actuated in order to expel the staples from the cartridge 124 towards the anvil 126 and, at the same time, to move a knife (not shown in the figures) from the proximal end region 128 to the distal end region 140 of the cartridge device 122. This knife is contained in the cartridge device 122 and runs in parallel to the rows of staples, wherein, in the embodiment, there are three rows of staples on each side of the knife. Again, further details of the knife and the actuating mechanism are given in U.S. Pat. No. 5,307,976.

The instrument 100 is used in a similar way as a conventional stapler. Tissue parts to be stapled (and cut) are introduced between the first tissue interface 130 and the second tissue interface 132 when the anvil 126 is moved away from the cartridge device 122. Then the anvil 126 is closed by moving it towards the cartridge device 122 via the lever 152, thus gripping and clamping the tissue between the first tissue interface 130 and the second tissue interface 132. In contrast to a conventional stapler, the tissue does not remain flat, but adapts to the wavy shape of the first tissue interface 130 and the second tissue interface 132. In this way, the tissue length effected by the jaw assembly 120 can be longer than the linear length of the tissue interfaces 130, 132. This allows the manipulation of a given length of tissue by an effectively smaller jaw assembly 120.

When the firing trigger 154 is actuated, the staples are expelled from the cartridge 124, penetrate the tissue and are formed by means of the anvil 126. Almost simultaneously the knife moves from the proximal end region 128 to the distal end region 140 of the cartridge device 122 and sequentially cuts the tissue along a line in-between the rows of staples.

The staple expelling mechanism disclosed in U.S. Pat. No. 5,307,976 drives the staples in a direction which is essentially perpendicular with respect to the longitudinal axis of the jaw assembly. The same type of mechanism could be used in the instrument 100. If it is preferred, however, that the staples are driven perpendicularly with respect to the local slope of the wavy shape of the first tissue interface 130, the mechanism has to be modified somewhat.

FIG. 3 shows an embodiment of another surgical stapling instrument 200. The design of the instrument 200 is similar to that of the stapling instrument disclosed in U.S. Pat. No. 4,633,874.

The instrument 200 comprises a body portion 210 with integrated handle and a jaw assembly 220. Similar to the instrument 100 described above, the jaw assembly 220 includes a cartridge device 222, which comprises a first tissue interface 230, and an anvil 226, which comprises a second tissue interface 232. In the embodiment, the cartridge device 222 contains four parallel straight rows of staples and a knife running along the center line of the staple arrangement for sequentially cutting tissue gripped between the cartridge device 222 and the anvil 226.

The first tissue interface 230 and the second tissue interface 232 have a wavy shape, like in the instrument 100.

To operate the instrument 200, the anvil 226 is moved towards the cartridge device 222 in a parallel relationship by means of a lever 252 in order to grip or clamp tissue between the cartridge device 222 and the anvil 226. In the state shown in FIG. 3, the lever 252 is swivelled in a position parallel to the body portion 210. Then an actuator 254 is pushed along the body portion 210 in distal direction, which causes the staples to be expelled from the cartridge device 222 for penetrating the tissue, the pointed ends of the staples being bent by the anvil 226, and to move the knife from the proximal end region 228 to the distal end region 240 of the cartridge device 222 for sequentially cutting the tissue.

FIG. 4 is an overall view of a surgical instrument 300 according to a third embodiment. The instrument 300 includes a handle section 310, a shaft 312 and a jaw assembly 320 at the distal end of the shaft 312. FIG. 5 is a magnified view of the jaw assembly 320.

The jaw assembly 320 comprises a cartridge device 322 and an anvil 326. Whereas in the instruments 100 and 200, the respective cartridge device and anvil are aligned in parallel to the longitudinal axis of the instrument, the anvil 326 and the cartridge device 322 extend transversally with respect to the shaft 312. The instrument 300 is designed for open surgery. The anvil 326 is rigidly connected to an extension of shaft 312, and the cartridge device 322 can be moved towards and away from the anvil 326, while a parallel relationship between a first tissue interface 330 provided at the cartridge device 322 and a second tissue interface 332 provided at the anvil 326 is maintained.

As in the previous embodiments, the first tissue interface 330 and the second tissue interface 332 have a wavy shape, in contrast to comparable instruments of the prior art. An example of such prior art instrument is described in U.S. Pat. No. 4,527,724 which also discloses details on the actuating elements of the instrument.

1. A surgical instrument, comprising
a frame having a body portion (110, 112; 210; 310, 312) and a handle (150; 210; 350),
a jaw assembly (120; 220; 320) in the distal region of the instrument (100; 200; 300), the jaw assembly (120; 220; 320) having a generally straight first tissue interface (130; 230; 330), a generally straight second tissue interface (132; 232; 332) and a tissue effector, and the jaw assembly (120; 220; 320) being adapted to grip tissue of a patient between the first tissue interface (130; 230; 330) and the second tissue interface (132; 232; 332) and to penetrate the gripped tissue by the tissue effector,
a moving device (152; 252; 352) adapted to move the first tissue interface (130; 230; 330) relative to the second tissue interface (132; 232; 332), and
an actuating device (154; 254; 354) adapted to actuate the tissue effector,
characterized in that both the first tissue interface (130; 230; 330) and the second tissue interface (132; 232; 332) have a generally wavy shape, the crests (134) and valleys (135) of the first tissue interface (130; 230; 330) generally matching the valleys (136) and crests (137), respectively, of the second tissue interface (132; 232; 332).

2. Surgical instrument according to claim 1, characterized in that the surgical instrument (100; 200; 300) is a stapling instrument, wherein the jaw assembly (120; 220; 320) includes a cartridge device (122; 222; 322) having the first tissue interface (130; 230; 330) and an anvil (126; 226; 326) having the second tissue interface (132; 232; 332), the tissue effector comprising, associated to the cartridge device (122; 222; 322), at least one generally straight row of staples which, upon actuation of the actuating device (154; 254; 354), exit from the first tissue interface (130; 230; 330), the second tissue interface (132; 232; 332) being adapted to form the ends of the staples.

3. Surgical instrument according to claim 2, characterized in that the line defining the row of staples follows the wavy shape of the first tissue interface (130; 230; 330).

4. Surgical instrument according to claim 2 or 3, characterized in that the tissue effector comprises a knife, which is contained in the cartridge device (122; 222; 322), runs generally in parallel to the row of staples and, upon actuation of the actuating device (154; 254; 354), moves towards the anvil (126; 226; 326).

5. Surgical instrument according to claim 4, characterized in that the knife has a wavy cutting edge, which, preferably, generally runs in parallel to the wavy shape of the first tissue interface (330).

6. Surgical instrument according to claim 2 or 3, characterized in that the tissue effector comprises a knife, which is adapted to move, upon actuation of the actuating device (154; 254), from one end region (128; 228) of the cartridge device (122; 222) to the opposite end region (140; 240) of the cartridge device (122; 222), generally in parallel to and along the row of staples, in order to sequentially cut the tissue gripped between the first tissue interface (130; 230) and the second tissue interface (132; 232).

7. Surgical instrument according to one of claims 4 to 6, characterized in that there is at least one row of staples on each side of the knife.

8. Surgical instrument according to one of claims 2 to 7, characterized in that the anvil is removable.

9. Surgical instrument according to one of claims 2 to 8, characterized in that the cartridge device (122; 222; 322) comprises a removable cartridge (124) containing the staples.

10. Surgical instrument according to claim 1, characterized in that the surgical instrument is a cutting instrument, wherein the jaw assembly includes a first gripping jaw having the first tissue interface and a second gripping jaw having the second tissue interface, the tissue effector comprising a knife, which is adapted to cut, upon actuation of the actuating device, the tissue gripped between the first tissue interface and the second tissue interface.

11. Surgical instrument according to claim 10, characterized in that the knife is generally straight, generally extends along the jaw assembly, and, upon actuation of the actuating device, moves from the first tissue interface towards the second tissue interface.

12. Surgical instrument according to claim 11, characterized in that the knife has a wavy cutting edge, which, preferably, generally runs in parallel to the wavy shape of the first tissue interface.

13. Surgical instrument according to claim 10, characterized in that the knife is adapted to move, upon actuation of the actuating device, from one end region of the gripping jaws to the opposite end region of the gripping jaws, generally along the gripping jaws, in order to sequentially cut the tissue gripped between the first tissue interface and the second tissue interface.

14. Surgical instrument according to one of claims 1 to 13, characterized in that the moving device (152; 252; 352) is adapted to move the first tissue interface (130; 230; 330) relative to the second tissue interface (132; 232; 332) in a generally parallel relationship.

15. Surgical instrument according to one of claims 1 to 14, characterized in that the body portion (310, 312) of the frame includes a shaft (312) and in that the longitudinal axes of the first tissue interface (330) and the second tissue interface (332) run transversely with respect to the longitudinal axis of the shaft (312).

16. Surgical instrument according to one of claims 1 to 14, characterized in that the body portion (110, 112; 210) of the frame includes a shaft (112; 210) and in that the longitudinal axes of the first tissue interface (130; 230) and the second tissue interface (132; 232) run generally in parallel with respect to the longitudinal axis of the shaft (112; 210).

17. Surgical instrument according to one of claims 1 to 16, characterized in that the surgical instrument is an endoscopic instrument (100).

18. Surgical instrument according to one of claims 1 to 17, characterized in that the jaw assembly (120) is removably mounted in the distal end region (116) of the body portion (110; 112).

19. Jaw assembly, which is adapted to the surgical instrument (100) according to claim 18.

20. Anvil, which is adapted to the surgical instrument according to claim 8.

21. Cartridge, which is adapted to the surgical instrument (100; 200; 300) according to claim 9.
